(19) United States
(12) Patent Application Publication
(10) Pub. No.: US 2019/0125462 A1
(43) Pub. Date: May 2, 2019

(54) MULTI-INPUT ROBOTIC SURGICAL SYSTEM CONTROL SCHEME

(71) Applicant: Covidien LP, Mansfield, MA (US)

(72) Inventors: William Peine, Ashland, MA (US); Peter Vokrot, Malden, MA (US)

(21) Appl. No.: 16/306,764

(22) PCT Filed: May 26, 2017
(86) PCT No.: PCT/US2017/034607

§ 371 Date: Dec. 3, 2018

Related U.S. Application Data
(60) Provisional application No. 62/345,032, filed on Jun. 3, 2016.

(51) Int. Cl. A61B 34/37; A61B 34/00; A61B 90/00

(52) U.S. Cl. CPC A61B 34/37; A61B 34/74; A61B 90/37; A61B 34/76

(57) ABSTRACT

Methods and systems are provided of operating a dual console robotic surgical system. The method includes receiving an input from a first console of the dual console robotic surgical system via a first input handle to move a robotic arm of the robotic surgical system. In response to receiving the input from the first input handle, the robotic arm is moved. Additionally, substantially simultaneously with the moving of the first input handle, an output is provided to thereby move a second input handle of a second console of the dual console robotic surgical system in substantially the same motion as the first input handle.

### CROSS-REFERENCE TO RELATED APPLICATIONS

This application claims the benefit of and priority to U.S. Provisional Application No. 62/345,032, filed on Jun. 3, 2016, the entire contents of which are incorporated by reference herein.

### TECHNICAL FIELD

The present disclosure relates to robotic surgical systems and, more particularly, to systems and methods for controlling multiple consoles included in a robotic surgical system.

### BACKGROUND

In order to become sufficiently skilled to perform surgical operations on patients, a surgeon may endure many hours of education and training in order to become an expert at performing an operation. For example, in addition to numerous hours of classroom training, surgeons are exposed to many hands-on training sessions as well. Specifically, a novice surgeon may spend many weeks and/or months in an operating room standing over and observing an expert surgeon. After an appropriate amount of observation time, the novice surgeon may be allowed to perform individual steps of the surgical procedure, which may, over time, build the novice surgeon's skills such that the novice surgeon is capable of performing an entire surgical procedure. Additionally or alternatively, the expert surgeon may place his or her hand over the novice surgeon's hand to guide the novice surgeon to appropriate positioning. As each particular surgical procedure involves different parts of an anatomy of a patient, the novice surgeon will typically receive extensive training on each different surgical procedure within the surgeon's specialty.

The training for surgical operations performed using robotic surgical procedures is no different. In many instances, while a patient is placed on a platform adjacent a robotic system, a novice surgeon stands over an expert surgeon who is positioned at a console remote from the robotic system. The expert surgeon provides input to a user interface at the console to thereby control the robotic system, for example, by using an input controller or handle to manipulate a tool coupled to an arm of the robotic system, such as an end effector or surgical instrument, to perform surgical operations on the patient. To provide the novice surgeon with a full training experience, the expert surgeon may switch positions with the novice surgeon to allow the novice surgeon to deliver an input into the console.

Although hands-on experience is an important aspect of a well-rounded training program, providing access to such experience can be challenging for certain procedures. For example, time may be of the essence in some surgical procedures, and hence, having the novice surgeon switch positions with the expert surgeon during the procedure may not be feasible. In other cases, certain steps of the procedure may not be handed off to another surgeon mid-procedure. As such, there is a need for improved systems and methods for training on robotic surgical systems.

### SUMMARY

The present disclosure generally relates to robotic surgical systems, non-transitory computer readable media, and methods of operating a dual console robotic surgical system. In an aspect of the present disclosure, a robotic surgical system includes a first console including a first input handle, a second console including a second input handle, a robotic arm, and a controller coupled to the first console, the second console, and the robotic arm. The controller includes a processor, and a memory coupled to the processor. The memory stores instructions that, when executed by the processor, cause the controller to receive an input from the first console via the first input handle to cause the robotic arm to move, in response to receiving the input from the first input handle, move the robotic arm, and substantially simultaneously with the moving of the first input handle, provide an output to thereby cause the second input handle of the second console to move in substantially the same motion as the first input handle.

In another aspect of the present disclosure, the robotic surgical system includes further instructions that when executed by the processor, cause the controller to provide a signal to the second console to provide a force feedback, in response to receiving an input from the second input handle.

In another aspect of the present disclosure, the first console and the second console are substantially identically configured. In still another aspect of the present disclosure, each of the first console and the second console has a corresponding base, and positioning of each of the input handles of each of the first console and the second console is based on a fixed coordinate frame relative to its corresponding base. In still another aspect of the present disclosure, each of the first console and the second console has a plurality of support arms, and positioning of the input handles of the each of the first console and the second console is based on positioning of a first support arm of the first console relative to a second support arm of the first console and a first support arm of the second console relative to a second support arm of the second console.

In another aspect of the present disclosure, the first console and the second console are not substantially identically configured and each of the first console and the second console has an input handle, and positioning of each of the input handles of each of the first console and the second console is based on Cartesian coordinates of each of the input handles.

In another aspect of the present disclosure, further instructions are included that, when executed by the processor, cause the controller to detect a movement of the second input handle in a direction that is not substantially identical to a movement of the first input handle, and in response to detecting the movement of the second input handle, increase a stiffness output by the second input handle. In still another aspect of the present disclosure, the stiffness output increases based on an increase in a distance between a movement path of the first input handle and a movement path of the second input handle.

In another aspect of the present disclosure, further instructions are included that, when executed by the processor, cause the controller to detect a movement of the second input handle in a direction that is not substantially identical to a movement of the first input handle, and in response to detecting the movement of the second input handle, increase a torque output by the second input handle.

In another aspect of the present disclosure, the memory further comprises instructions that, when executed by the processor, cause the controller to detect a movement of the second input handle in a direction that is not substantially identical to a movement of the first input handle, and in response to detecting the movement of the second input handle, overriding the movement of the second input handle to thereby move the robotic arm according to the movement of the first input handle.

According to another aspect of the present disclosure, a method of operating a dual console robotic surgical system is provided. The method includes receiving an input from a first console of the dual console robotic surgical system via a first input handle to move a robotic arm of the robotic surgical system, in response to receiving the input from the first input handle, moving the robotic arm, and substantially simultaneously with the moving of the first input handle, providing an output to thereby move a second input handle of a second console of the dual console robotic surgical system in substantially the same motion as the first input handle.

In another aspect of the present disclosure, the method further includes providing a signal to the second console of the dual console robotic surgical system to provide a force feedback, in response to receiving an input from the second input handle.

In another aspect of the present disclosure, the method further includes detecting a movement of the second input handle in a direction that is not substantially identical to a movement of the first input handle, and in response to detecting the movement of the second input handle, increasing a stiffness output by the second input handle. In still another aspect, the method further includes increasing the stiffness output increases based on an increase in a distance between a movement path of the first input handle and a movement path of the second input handle.

In another aspect of the present disclosure, the method further includes detecting a movement of the second input handle in a direction that is not substantially identical to a movement of the first input handle, and in response to detecting the movement of the second input handle, increasing a torque output by the second input handle.

In another aspect of the present disclosure, the first console is a main console and the second console is an auxiliary console.

In another aspect of the present disclosure, the method further includes detecting a movement of the second input handle in a direction that is not substantially identical to a movement of the first input handle, and in response to detecting the movement of the second input handle, overriding the movement of the second input handle to thereby move the robotic arm according to the movement of the first input handle.

According to yet another aspect of the present disclosure, a non-transitory computer readable medium is provided storing instructions for operating a dual console robotic surgical system, the instructions that, when executed by a processor, cause the processor to receive an input from a first console of the dual console robotic surgical system via a first input handle of the first console to move a robotic arm, in response to receiving the input from the first input handle, move the robotic arm, and substantially simultaneously with the moving of the first input handle, provide an output to thereby move a second input handle of a second console of the dual console robotic surgical system in substantially the same motion as the first input handle.

In another aspect of the present disclosure, the non-transitory computer readable medium further includes instructions that, when executed by a processor, cause the processor to provide a signal to the second console of the dual console robotic surgical system to provide a force feedback, when an input is received from the second input handle.

According to still yet another aspect of the present disclosure, a robotic surgical system includes a first console including a first input handle, a second console including a second input handle, a robotic arm including a surgical tool configured to be disposed adjacent to a surgical site, and a controller coupled to the first console, the second console, and the robotic arm. The controller includes a processor, and a memory coupled to the processor, the memory storing instructions that, when executed by the processor, cause the controller to receive an input from the first console via the first input handle to move the surgical tool to a location within the surgical site, determine a coordinate of the location of the surgical tool within the surgical site, and provide an output to move the second input handle of the second console to a position that translates to the location of the surgical tool within the surgical site.

In another aspect of the present disclosure, the memory further includes instructions that, when executed by the processor, cause the controller to obtain a position of the surgical instrument relative to a base of the robotic arm to which the surgical instrument is coupled.

In another aspect of the present disclosure, the robotic surgical system further includes an imaging device coupled to the controller, where the imaging device is configured to be disposed over the surgical site, wherein the memory further includes instructions that, when executed by the processor, cause the controller to obtain a position of the surgical instrument from an image of the surgical site acquired by the imaging device.

Further details and aspects of exemplary embodiments of the present disclosure are described in more detail below with reference to the appended figures.

### BRIEF DESCRIPTION OF THE DRAWINGS

Various aspects of the present disclosure are described hereinbelow with reference to the drawings, which are incorporated in and constitute a part of this specification, wherein:

FIG. 1 is a schematic illustration of a robotic surgical system in accordance with the present disclosure;

FIG. 2 is a perspective view of an input device of the robotic surgical system of FIG. 1, in accordance with the present disclosure;

FIG. 3 is a close up view of a portion of the input device of FIG. 2, in accordance with the present disclosure;

FIG. 4 is a functional block diagram of the system architecture for controlling the multi-input robotic surgical system of FIG. 1;

FIG. 5 is a block diagram of the control components for controlling the multi-input robotic surgical system of FIG. 1;

FIG. 6 is a flow diagram of a process for controlling the robotic surgical system of FIG. 1;

FIG. 7 is a flow diagram of a illustrating operation of the robotic surgical system of FIG. 1 in a lock-out mode, in accordance with an embodiment;

FIG. 8 is a flow diagram of a illustrating operation of the robotic surgical system of FIG. 1 in a lock-out mode, in accordance with another embodiment;

FIG. 9 is a flow diagram of a illustrating operation of the robotic surgical system of FIG. 1 in a non lock-out mode, in accordance with an embodiment; and

FIG. 10 is a flow diagram of a illustrating operation of the robotic surgical system of FIG. 1 in a non lock-out mode, in accordance with another embodiment.

### DETAILED DESCRIPTION

Embodiments of the present disclosure are now described in detail with reference to the drawings in which like reference numerals designate identical or corresponding elements in each of the several views. As used herein, the term “clinician” refers to a doctor, a nurse, or any other care provider and may include support personnel. Throughout this description, the term “proximal” refers to the portion of the device or component thereof that is farthest away from the patient and the term “distal” refers to the portion of the device or component thereof that is closest to the patient.

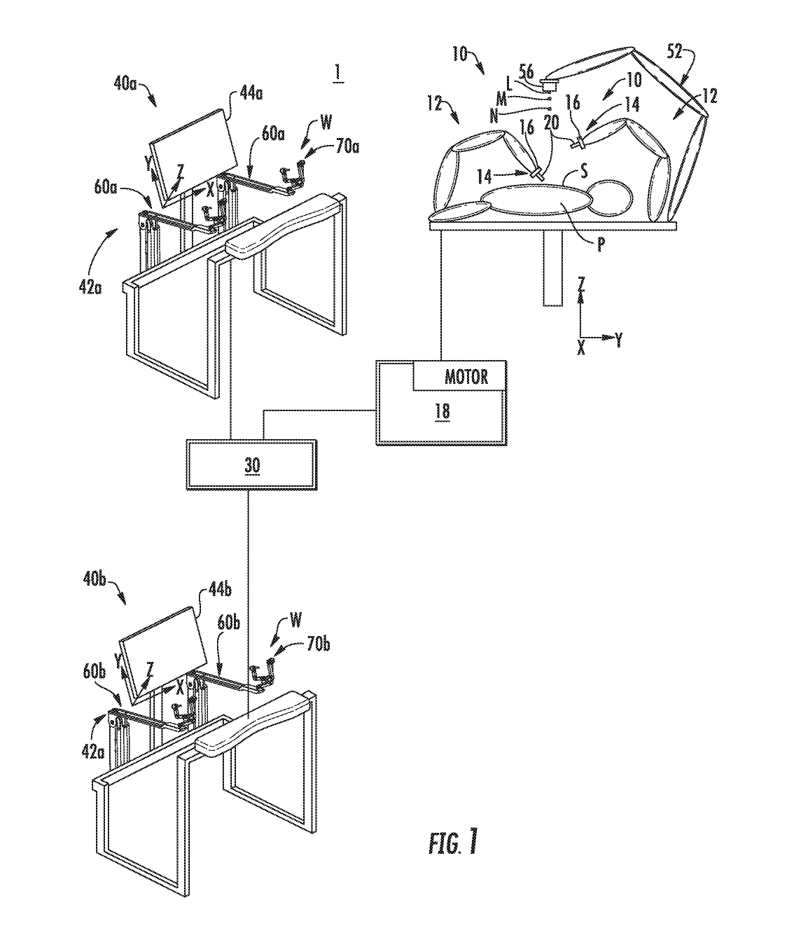

Referring to FIG. 1, a multi-input robotic surgical system 1 in accordance with the present disclosure is shown generally and includes a robotic system 10, a processing unit 30, and user interfaces 40a, 40b (collective referred to below as “user interfaces 40”). The robotic system 10 generally includes robotic arms 12 and a robot base 18. Each of the robotic arms 12, which may be in the form of linkages, has an end 14 that moveably supports an end effector, instrument or tool 20 configured to act on tissue. The ends 14 of the robotic arms 12 may include an imaging device 16 for imaging a surgical site “S”.

Each user interface 40 communicates with the robot base 18 through the processing unit 30 and includes a display device 44a, 44b (collectively referred to below as “display devices 44”) which is configured to display images. In accordance with an embodiment, the display devices 44 display three-dimensional images of the surgical site “S” which may include data captured by imaging devices 16 and/or include data captured by imaging devices (not shown) that are positioned about the surgical theater (e.g., an imaging device positioned within the surgical site “S”), an imaging device positioned adjacent the patient “P”, or an imaging device 56 positioned at a distal end of an imaging arm 52). The imaging devices (e.g., imaging devices 16, 56) may capture visual images, infra-red images, ultrasound images, X-ray images, thermal images, and/or any other known real-time images of the surgical site “S”. The imaging devices transmit captured imaging data to the processing unit 30 which creates three-dimensional images of the surgical site “S” in real-time from the imaging data and transmits the three-dimensional images to the display devices 44 for display. In another embodiment, the displayed images are two-dimensional renderings of the data captured by the imaging devices.

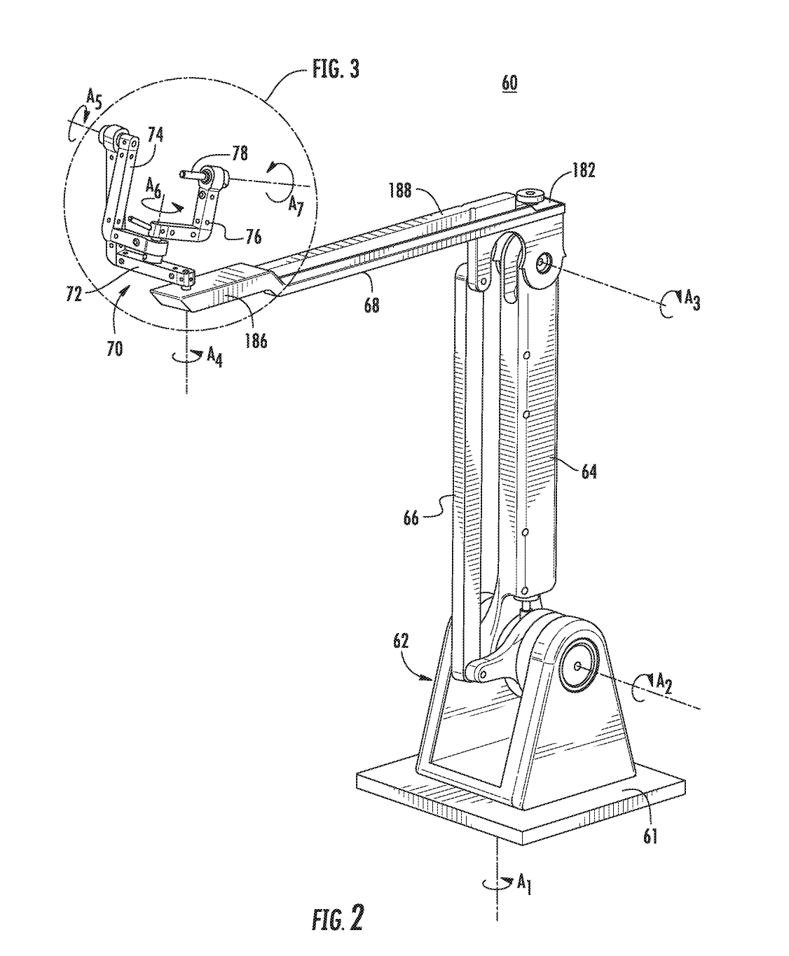

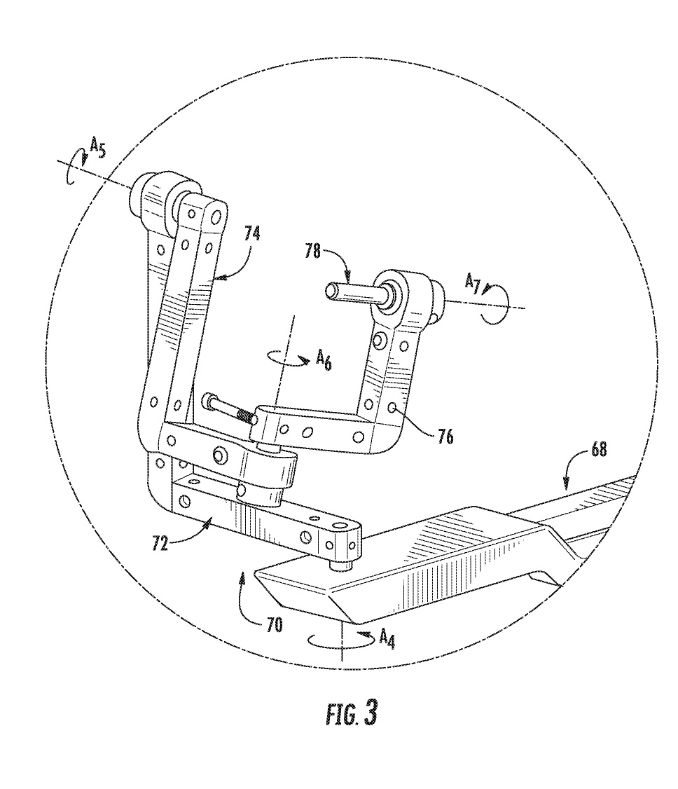

Each user interface 40 also defines a workspace “W” and includes input handles attached to gimbals 70a, 70b (also collectively referred to and shown as gimbals 70 in FIGS. 2 and 3) which allow a surgeon to manipulate the robotic system 10 (e.g., move the robotic arms 12, the ends 14 of the robotic arms 12, and/or the tools 20). Each of the gimbals 70 is in communication with the processing unit 30 to transmit control signals thereto and to receive feedback signals therefrom. Additionally or alternatively, each of the gimbals 70 may include control interfaces or input devices (not shown) which allow the surgeon to manipulate (e.g., clamp, grasp, fire, open, close, rotate, thrust, slice, etc.) the tools 20 supported at the ends 14 of the robotic arms 12.

Each of the gimbals 70 is moveable to move the ends 14 of the robotic arms 12 and/or to manipulate tools 20 within a surgical site “S”. The three-dimensional images on the display device 44 are orientated such that movement of the gimbals 70 moves the ends 14 of the robotic arms 12 and/or the tools 20 as viewed on the display device 44. It will be appreciated that the orientation of the three-dimensional images on the display device may be mirrored or rotated relative to view from above the patient “P”. In addition, it will be appreciated that the size of the three-dimensional images on the display device 44 may be scaled to be larger or smaller than the actual structures of the surgical site “S” permitting the surgeon to have a better view of structures within the surgical site “S”. As the gimbal 70 is moved, the tools 20 are moved within the surgical site “S”. Movement of the tools 20 may also include movement of the ends 14 of the robotic arms 12 which support the tools 20. Although illustrated as a handle, a handle including a clutch switch, one or more of the input devices 42 include and are not limited to, e.g., a touchpad, joystick, keyboard, mouse, or other computer accessory, and/or a foot switch, pedal, trackball, or other actuatable device configured to translate physical movement from the clinician to signals sent to the processing unit 30.

Referring to FIG. 2, each control arm 60 of the user interface 40 (FIG. 1) includes a rotatable base member 62, a vertical member 64, a support member 66, a horizontal member 68, and a gimbal 70. The rotatable base member 62 is rotatably supported on a fixed base 61. The control arms 60 of the user interface 40 may each be supported on the same fixed base 61 or each of the control arms 60 of a user interface 40 may be supported on a separate fixed base 61. The fixed base 61 may be rolled or otherwise moveable about a surgical environment before or after a surgical procedure and be fixed in position during a surgical procedure. The horizontal member 68 extends from a first end 182 to a second end 186 thereof and includes a rib 188 disposed along a centerline thereof between the first and second ends 182, 186. The second end 186 of the horizontal member 68 rotatably supports the gimbal 70.

With additional reference to FIG. 3, the gimbal 70 includes a support arm 72, a swing arm 74, an input support arm 76, and an input shaft 78. Each of the support, swing, and support arms 72, 74, 76 are L-shaped having a horizontal portion and a vertical portion. The support, swing, and support arms 72, 74, 76 are sized such that the arms 72, 74, 76 nest within each other when aligned in a single plane. For example, the input support arm 76 nests in the swing arm 74, which is nested in the support arm 72. The input shaft 78 is engageable with an adaptor or input device (not shown) to control functions of the tool 20 (FIG. 1) of the robotic system 10.

The control arm 60 is rotatable about seven axes of rotation in response to a surgeon interfacing with the gimbal 70 (e.g., interfacing with an input device disposed on the input shaft 78). Movement of the control arm 60 about the seven axes of rotation is detected by the processing unit 30 (FIG. 1) to manipulate the robotic arms 12 and tools 20 of the robotic surgical system 1. The construction of the control arm 60 and gimbal 70 allows movement of the respective members and arms to rotation about the seven axes of rotation.

The movement of the tools 20 are scaled relative to the movement of the input handles, and hence, the control arm 60 and gimbals 70. When the input handles are moved within a predefined workspace “W”, the input handles send control signals to the processing unit 30. The processing unit 30 analyzes the control signals to move the tools 20 in response to the control signals. The processing unit 30 transmits scaled control signals to the robot base 18 to move the tools 20 in response the movement of the input handles.

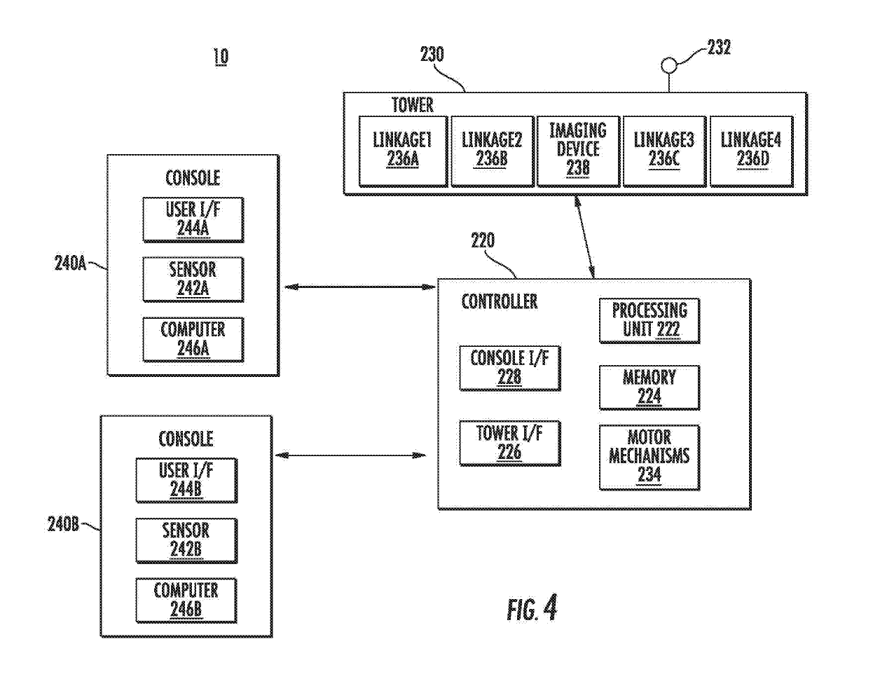

FIG. 4 is a functional block diagram of the robotic surgical system 1 of FIG. 1. The robotic surgical system 1 effects the movement of the robotic arms 18 and/or tools 20 via the input handles of the user interface 40. In this regard, the robotic surgical system 1 includes a controller 220, a tower 230, and consoles 240a, 240b. The controller 220 is configured to communicate with the tower 230 to thereby provide instructions for operation, in response to input received from one of the consoles 240a, 240b.

The controller 230 generally includes a processing unit 222, a memory 224, a tower interface 226, and a consoles interface 228. The processing unit 222, in particular by means of a computer program stored in the memory 224, functions in such a way to cause components of the tower 230 to execute a desired movement according to a movement defined by input devices 242 of the consoles 240a, 240b. In this regard, the processing unit 222 includes any suitable logic control circuit adapted to perform calculations and/or operate according to a set of instructions. The processing unit 222 may include one or more processing devices, such as a microprocessor-type of processing device or other physical device capable of executing instructions stored in the memory 224 and/or processing data. The memory 224 may include transitory type memory (e.g., RAM) and/or non-transitory type memory (e.g., flash media, disk media, etc.). The tower interface 226 and consoles interface 228 communicate with the tower 230 and consoles 240, respectively, either wirelessly (e.g., Wi-Fi, Bluetooth, LTE, etc.) and/or via wired configurations. Although depicted as separate modules, the interfaces 226, 228 are a single component in other embodiments.

The tower 230 includes a communications interface 232 configured to receive communications and/or data from the tower interface 226 for manipulating motor mechanisms 234 to thereby move the robotic arms 236a-d. In accordance with an embodiment, the motor mechanisms 234 are configured to, in response to instructions from the processing unit 222, receive an application of current for mechanical manipulation of cables (not shown) which are attached to the arms 236a-d to cause a desired movement of a selected one of the arms 236a-d and/or an instrument coupled to an arm 236a-d. The tower 230 also includes an imaging device 238, which captures real-time images and transmits data representing the images to the controller 230 via the communications interface 232.

To manipulate the devices of the tower 230, each console 240a, 240b has an input device 242a, 242b, a display 244a, 244b, and a computer 246a, 247b. Each input device 242a, 242b is coupled to the corresponding computer 246a, 246b and is used by the clinician to provide an input. In this regard, the input device 242a, 242b may be a handle or pedal, or a computer accessory, such as a keyboard, joystick, mouse, button, touch screen, switch, trackball or other component. The display 244a, 244b displays images or other data received from the controller 220 to thereby communicate the data to the clinician. The computer 246a, 246b includes a processing unit and memory, which includes data, instructions and/or information related to the various components, algorithms, and/or operations of the tower 230 and can operate using any suitable electronic service, database, platform, cloud, or the like.

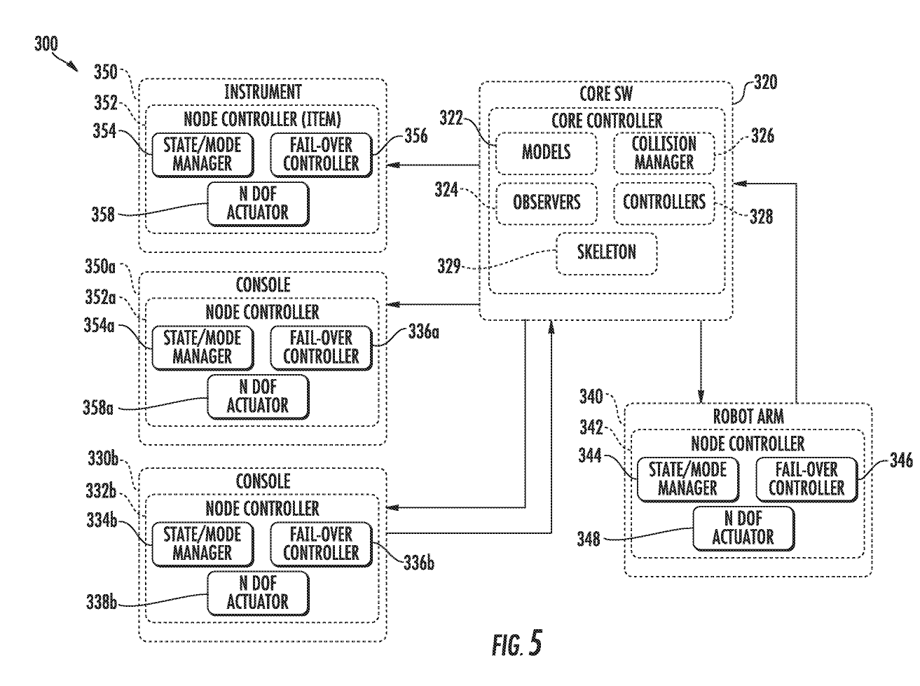

FIG. 5 is a simplified functional block diagram of a system architecture 300 of the robotic surgical system 1 included in FIG. 4. The system architecture 300 includes a core module 320, console modules 330a, 330b, a robot arm module 340, and an instrument module 350. The core module 320 serves as a central controller for the robotic surgical system 1 and coordinates operations of all of the other modules 330a, 330b, 340, 350. For example, the core module 320 maps control devices to the robotic arms 18, determines a current status of the system 10, performs all kinematics and frame transformations, and relays resulting movement commands. In this regard, the core module 320 receives and analyzes data from each of the other modules 330a, 330b, 340, 350 in order to provide instructions or commands to the other modules 330a, 330b, 340, 350 for execution within the robotic surgical system 1. Although depicted as separate modules, two or more of the modules 320, 330a, 330b, 340, and 350 are combined as a single component in other embodiments.

The core module 320 includes models 322, observers 324, a collision manager 326, controllers 328, and a skeleton 329. The models 322 include units that provide abstracted representations (base classes) for controlled components, such as the motors 18 and/or the robotic arms 12. The observers 324 create state estimates based on input and output signals received from the other modules 330a, 330b, 340, 350. The collision manager 326 prevents collisions between components that have been registered within the system 10. The skeleton 329 tracks the system 10 from a kinematic and dynamics point of view. For example, the kinematics item may be implemented either as forward or inverse kinematics, in an embodiment. The dynamics item may be implemented as algorithms used to model dynamics of the system's components.

Each console module 330a, 330b communicates with surgeon control devices at corresponding consoles 240a, 240b and relays inputs received from the console 240a, 240b to the core module 320. In accordance with an embodiment, each console module 330a, 330b communicates button status and control device positions to the core module 320 and includes a corresponding node controller 332a, 330b that includes a state/mode manager 334a, 334b, a fail-over controller 336a, 336b, and a N degree-of-freedom (“DOF”) actuator 338a, 338b.

The robot arm module 340 coordinates operation of a robot arm subsystem, an arm cart subsystem, a set up arm, and an instrument subsystem in order to control movement of a corresponding arm 12. It will be appreciated that the robot arm module 340 corresponds to and controls a single arm. As such, although a single robot arm module 340 is shown, additional modules 340 are included for each of the arms 236a-d, in an embodiment. Each robot arm module 340 includes a node controller 342, a state/mode manager 344, a fail-over controller 346, and a N degree-of-freedom (“DOF”) actuator 348.

The instrument module 350 controls movement of a tool 20 (shown in FIG. 2) attached to the arm 12 (also shown in FIG. 2). The instrument module 350 is configured to correspond to and control a single tool. Thus, in configurations in which multiple tools are included, additional instrument modules 350 are likewise included. In an embodiment, the instrument module 350 obtains and communicates data related to the position of tool 20 on the arm 12. Each instrument module 350 has a node controller 352, a state/mode manager 354, a fail-over controller 356, and an N degree-of-freedom (“DOF”) actuator 358.

The system 1 is configured such that the consoles 240a, 240b operate concurrently. As a result, an expert clinician at a first console 240a can provide an input to manipulate the arms 236a-d in a desired manner, while a junior clinician at a second console 240b can grasp corresponding input handles to feel and mimic the expert clinician's motions. As such, in an embodiment, the first console 240a may be a main console and the second console 240b may be an auxiliary console.

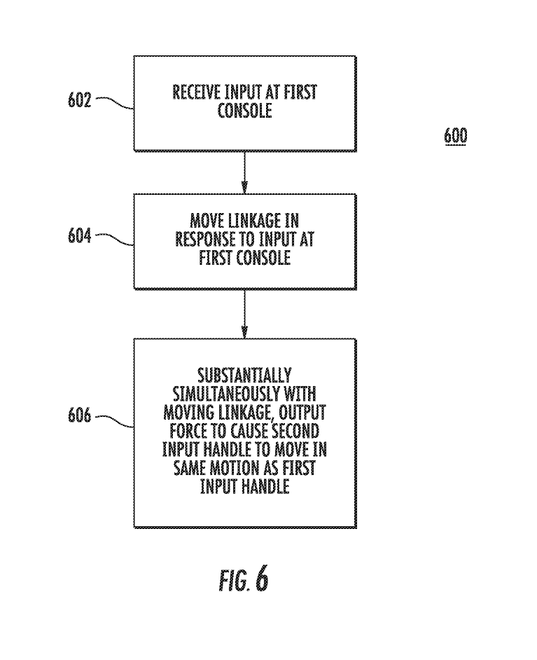

FIG. 6 is a flow diagram of a control process 600 for concurrent operation of a multi-input surgical robotic system. According to an embodiment, the process 600 includes receiving an input from a first console 240a via a first input handle 42a to move a selected portion of one of the robotic arms 236a-d at step 602. A force is exerted by the expert clinician on the first input handle 42a that is sufficient to cause the robotic arm itself, or a surgical instrument or tool 20 coupled to or extending from the robotic arm 236a-d, collectively referred to as a selected portion of the robotic arm, to move in a desired manner.

In response to receiving the input from the first input handle 42a, the selected portion of the robotic arm is moved at step 604. In an embodiment, the movement of the selected portion of the robotic arm is scaled relative to the force exerted against the first input handle 42a by the expert clinician. For example, the processing unit 30 transmits scaled control signals to the robot base 18 to move the tools 20 in response to the movement of the input handle 42a. In this regard, the processing unit 30 scales the control signals by dividing an Input_{distance} (e.g., the distance moved by the input device 42a) by a scaling factor S_{F} to arrive at a scaled Output_{distance} (e.g., the distance that one of the ends 14 is moved). In some instances one or more scaling factors “S_{F}” used in operation during a surgical procedure may be in a range between about 1 and about 10 (e.g., 3). Scaling may be represented by the following equation:

Output_{distance}=Input_{distance}/S_{F} [1]

It will be appreciated that the larger scaling factor “S_{F}”, the smaller the movement of the tools 20 will be relative to the movement of the input handle 42a. Thus, to facilitate repositioning of the input handle 42a relative to a corresponding surgical tool 20 to be driven, a larger scaling factor “S_{F}” may be used instead so that the tool 20 moves a distance that is much less than that traveled by the input handle 42a. In some instances this repositioning scaling factor may be at least about 100 or more.

In an alternative embodiment, both the scaling factors may be less than one (e.g. operating scaling factor is about 0.5 and repositioning scaling factor is 0.005) such that the scaling factors are multiplied by the input distance to calculate the output distance that the tools are moved. This scaling may be represented by the following equation:

Output_{distance}=Input_{distance}*S_{F} [2]

It will be appreciated that the scaling may be adjusted to the clinician's preference. In an embodiment, the ratio may be 1:1 so that the force provided by the clinician to the input handle 42a provides an output that matches that of the robotic arm, instrument or the tool. In this way, moving the input handle 42a, 42b provides a feel to the clinician that mimics holding and/or moving the tool with minimal intervening components.

At step 606, substantially simultaneously with the moving of the first input handle 42a, an output is provided to cause the second input handle 42b of the second console 240b to move in substantially the same motion as the first input handle 42a. As such, in an embodiment, when the novice clinician at the second console 240b places his or her hands onto the second input handle 42b, the novice clinician follows the movement of the second input handle 42b. Thus, while the procedure is being performed by the expert clinician at the first console 240a, the input handles 42a, 42b of the two consoles 240a, 240b move in a manner mirroring the movement as intended by the expert clinician on the first console 240a.

According to an embodiment, the system 1 includes a lock-out mode during which input provided to the second console 240b cannot effect movement of the selected portion of the robotic arm. As such, even if the novice clinician attempts to move the second input handle 42b, any input provided to the second console 240b is not provided to the controller 220.

In another embodiment in which the system 1 includes the lock-out mode, the system 10 is configured to receive an input to reposition the input handle 42b at the second console 240b, and in response to the received input, provide an output to counteract the input. In an embodiment, the output is a signal to the second console 240b to provide a force feedback to the novice clinician via the input handle 42b of the second console 240b to return to an intended location.

The lock-out mode may be implemented into any one of numerous system configurations. In an example configuration of system 10, both of the user interfaces 40 and consoles 240a, 240b are substantially identical to each other. In an embodiment, the positioning of each of the input handles 42a, 42b is based on a fixed coordinate frame relative to its corresponding fixed base 61. Here, when an input is detected at the first input handle 42a at the first console 240a, signals are sent to the controller 230 indicating a plurality of coordinates making up a path along which the first input handle 42a travels, for example, the intended path. In an embodiment, an end of the path is an intended location of the first input handle 42a. In response to receiving the signals from the first console 240a, signals are sent by the controller 230 to the second console 240b to output a force or to provide a force feedback to effect movement of the second input handle 42a to positions at the same coordinates of the intended path of the first input handle 42a, except at the second console 240b. In another embodiment, the signal sent by the controller 230 to the second console 240b outputs a force to effect movement of the second input handle 42a to a position at the same coordinate as the intended location of the first input handle 42a, except at the second console 240b.

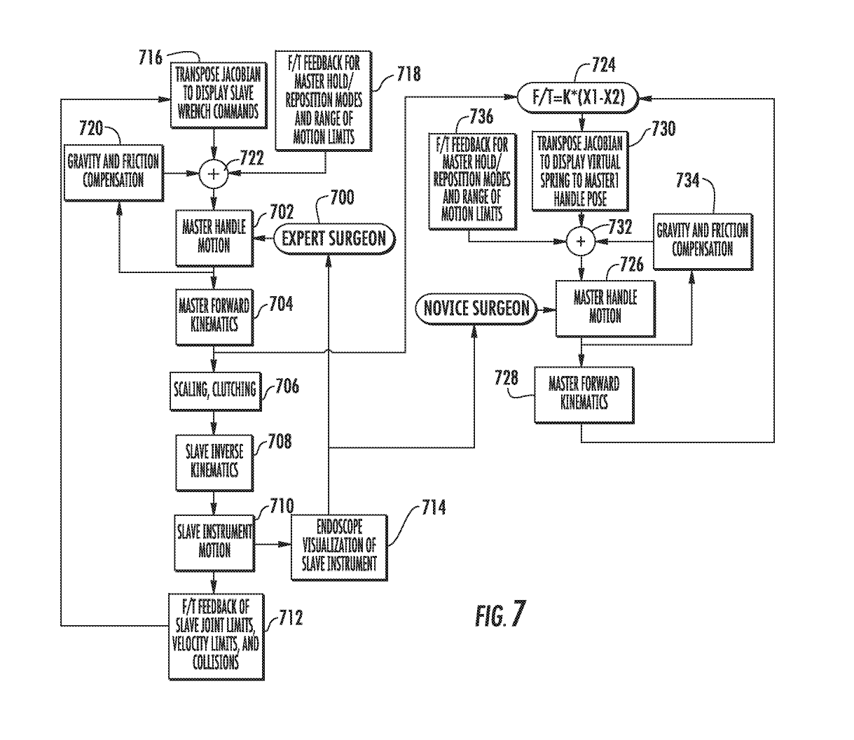

FIG. 7 is a flow diagram illustrating operation of the system 1 in a lock-out mode, according to an embodiment. The operation of the system 1 includes positioning input handles 42a, 42b based on a fixed coordinate frame, according to an embodiment. Such an embodiment may be implemented in a configuration in which the first and second consoles 240a, 240b are not identically situated. The positioning of the input handles 42a, 42b as illustrated in FIG. 7 is based on matching the pose and roll, pitch, and yaw of the input handles 42a, 42b.

Beginning at block 700 with the expert clinician, an input is provided to effect a master handle motion, for example, by using the first input handle 42a at the first console 240a, at block 702. Joint angles of the master input device (q1) are measured from the input allowing forward kinematics of the input to be obtained at 704 to output a handle pose of the first input handle 42a (X1). Based on the handle pose of the first input handle 42a, scaling and clutching are applied at block 706 to thereby output a desired instrument pose. The scaling and/or clutching may be pre-set by the expert clinician, depending on the expert clinician's preference or may be included as a factory-installed parameter. The applied scaling and clutching are used in determining the positioning of the tool 20, which is to be controlled using the first input handle 42a.

At block 708 slave inverse kinematics are calculated, outputting the desired slave joint angles. In particular, inverse kinematics are used to determine the joint angles at which to position the tool 20 to be controlled using the first input handle 42a. The desired slave joint angles are used to effect the slave instrument motion at block 710, for example, by moving the tool 20 to be controlled accordingly, thereby outputting actual slave joint angles. Visual feedback of the actual position and orientation of the instruments for teleoperation is obtained using the endoscope visualization equipment at block 714, which is then processed into a three-dimensional high definition video feed provided to displays at the expert and novice clinician consoles, for example, the first and second consoles 240a, 240b.

In addition to visual feedback to the surgeon, a force/torque (F/T) feedback wrench is calculated in block 712 based on the actual slave joint angles output from block 710. Force feedback can be used to provide a haptic indication of the state of the slave robot and instruments under control, such as when the slave robot or instrument reaches a joint range of motion limit, exceeds allowed velocities, or experiences a collision. The force/torque (F/T) feedback of the slave joint limits, velocity limits, and collisions may be pre-set by the expert clinician, depending on the expert clinician's preference or may be included as a factory-installed parameter. The force and torque command (F/T or wrench) output from block 712 is then processed by block 716 using a transpose Jacobian function to calculate the required joint torques in the input device to display the desired slave wrench commands coming from block 712. The required input device joint torques needed to display the F/T wrench feedback for the first input handle 42 are then combined at 722 with the joint torques required for hold/reposition modes and range of motion limits from block 718, and gravity and friction compensation from block 720 (which was obtained when the master handle motion was performed at block 702). Hold/reposition modes and range of motion limits from block 718 are predetermined values, which may be pre-set or may be set by the expert clinician as desired parameters, and that define the physical limits of the space of the console (for example, the first console 240a at which the expert surgeon is located). In any case, as a result of the combined operations, the joint torques for the first input handle 42a are obtained at block 722 and taken into account when the expert clinician provides further input via the first input handle 42a at block 702.

To determine the placement of the second input handles 42b, inputs from the expert clinician console and the novice clinician console are used in an algorithm suitable to provide forces and torques to the novice surgeon through handles 42b that allow the novice surgeon to mimic the motions of the expert surgeon. One representative equation to obtain the force/torque aspect is shown as follows:

F/T=K*(X1−X2) [3]

where:

K is the spring constant of the system;

X1 is the first input handle pose; and

X2 is the second input handle pose.

Here, X1 obtained from block 704 is provided for the calculation at block 724, and X2 is obtained from the master handle motion by the novice clinician at block 726. Specifically, the master handle motion by the novice clinician is detected at block 726, for example, through input to the second input handle 42b of the second console 240b. The joint angles (q2) output at block 726 are then supplied for the calculation of master forward kinematics at block 728 to output the second input handle pose (X2) to be sent block 724 to complete the calculation. It should be appreciated by those skilled in the art that equation 3 is a simplification and the actual computation must address Cartesian forces in the x, y, and z directions in a different manner than torque in the roll, pitch, and yaw directions. The output of these algorithms will result in forces and torque being applied to the novice's handles 42b to drive the handle into the same pose (position and orientation) as the expert's handles pose.

No matter the particular algorithm used at block 724, at block 730, a transpose Jacobian function is then applied to the output of block 724 to calculate the required joint torques of the novice surgeon's input device to display a virtual spring to the handle pose of the first input handle 42a. These joint torques are then combined at block 732 with the required joint torques needed to implement gravity and friction compensation obtained at block 734 from the joint angles (q2), and with the joint torques required for master hold/reposition modes and range of motion limits at block 736. The joint torques from block 732 are taken into account for the handle motion of second input handle 42b at 726. In this way, movements input by the expert clinician via the input handle 42a at the first console 240a are experienced by the novice clinician at the input handle 42b of the second console 240b.

In another embodiment in which both user interfaces 40 and consoles 240a, 240b are substantially identical to each other, positioning of the input handles 42a, 42b is based on the positioning of selected portions of the input handles 42a, 42b relative to the other portions of the input handles 42a, 42b. For example, when an input representing a movement of a first support arm relative to a second support arm of the first input handle 42a is detected at the first console 240a (which may be an intended location of the first support arm), a signal is sent to the controller 230 representing the first joint position. The first support arm position is indicated as a degree difference relative to the second support arm, a distance from the second support arm, a force or a torque to be applied to move the first support arm away from the second support arm, coordinates indicating a start and an end position of the first support arm relative to the second support arm, and the like. In any case, a signal is then transmitted to the second console 240b to reposition the first support arm at the second input handle 42b in the same manner relative to the second support arm of the second input handle 42b.

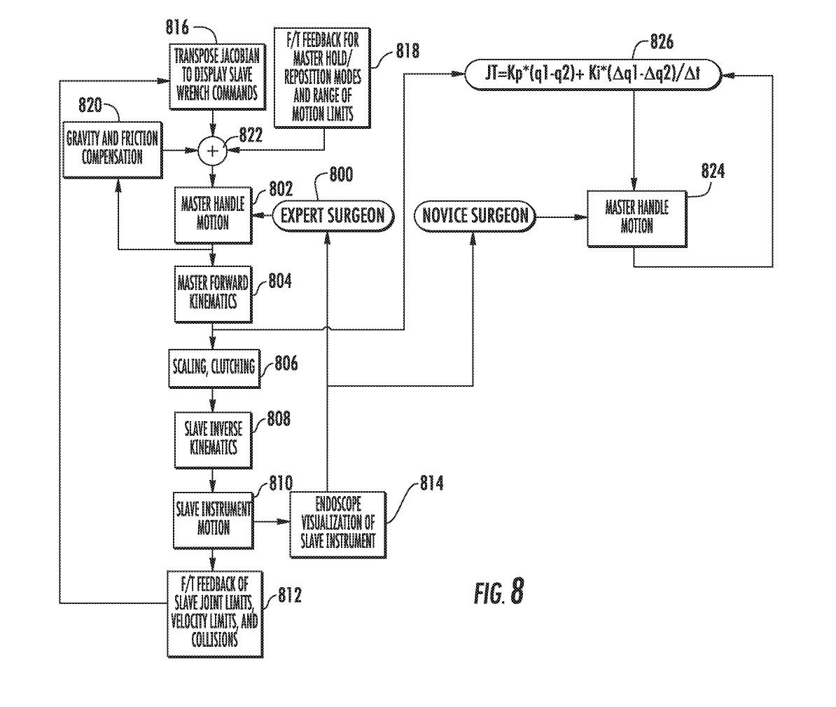

FIG. 8 is a flow diagram illustrating operation of the system 1 in another lock-out mode, according to an embodiment. Here, the operation of the system 1 includes positioning the input handles 42a, 42b based on the relative positioning of the joints of the input handles 42a, 42b, according to an embodiment. For example, in an embodiment in which the first and second consoles 240a, 240b are identically configured, joints (or support arms) of the input handles 42a, 42b correspond to each other such that movement of one joint of the first input handle 42a is identical to the movement of the same joint on the second input handle 42b.

With reference to block 800, the expert clinician provides an input to effect a master handle motion, for example, using the first input handle 42a of the first console 240a, at block 802. Joint angles of the master input device (q1) are measured from the input to allow forward kinematics of the input to be obtained at block 804. Based on the handle pose of the first input handle 42a, scaling and clutching are applied to the handle pose of the first input handle 42a at block 806 to output a desired instrument pose. Similar to the procedure above, the scaling and clutching may be pre-set by the expert clinician, depending on the expert clinician's preference or may be included as a factory-installed parameter.

At block 808 slave inverse kinematics are calculated. Specifically, the inverse kinematics corresponding to the tool 20 to be controlled using the first input handle 42a are used to determine the joint angles to which to position the tool to be controlled 20 using the first input handle 42a and the desired slave joint angles are used to effect the slave instrument motion at block 810, so that the tool 20 controlled by the first input handle 42a moves accordingly. The movement of the tool 20 at block 810 also affects the endoscope visualization of the slave instrument at block 814. For example, the movement is captured as three-dimensional high definition video, which is fed to displays viewable by the expert and novice clinicians at the first and second consoles 240a, 240b.

From the movement of the tool 20 at block 810, a force/torque feedback wrench is calculated at block 812 based on the actual slave joint angles output from block 810. The F/T feedback of the slave joint limits, velocity limits, and collisions may be pre-set by the expert clinician, depending on the expert clinician's preference or may be included as a factory-installed parameter. The force/torque command (F/T wrench) output from block 812 is processed at block 816 using a transpose Jacobian function to calculate the required joint torques in the input device to display the desired slave wrench commands from block 812. The required input device joint torques are then combined at block 822 with the joint torques required for hold/reposition modes and range of motion limits (which may be predetermined values that may be pre-set or set by the expert clinician as desired parameters) from block 818, and gravity and friction compensation from block 820 (obtained when the master handle motion was performed at block 802). As a result of the combining operation at block 822, the joint torques for the first input handle 42a are obtained and taken into account when the expert clinician provides additional input to the handles at block 802, which as noted above, outputs joint angles (q1).

When the second console 240b is in use, the inputs provided to the first console 240a via the first input handle 42a effect the movement of the joints of the second input handle 42b. In this regard, to determine the placement of the joints of the second input handle 42b, inputs from the expert clinician console and the novice clinician console are used in an algorithm suitable to provide the forces and torques to the second input handle 42b mimicking the motions of the expert clinician. One representative equation for such a calculation is shown as follows:

JT=Kp*(q1−q2) [4]

where:

Kp is a proportional spring constant;

q1 is the joint angle of the first input handle 42a; and

q1 is the joint angle of the second input handle 42b.

For example, the novice clinician provides an input via the second input handle 42b to effect handle motion at block 824, which outputs joint angles (q2). Using the joint angles obtained (q1) at block 804 and the joint angles (q2) output from block 824, the calculation from block 826 is then output and taken into account when the novice clinician provides additional input to the handles at block 824.

Another representative equation is provided below, to permit improved stability of the second input handle 42b:

JT=Kp*(q1−q2)+Kd*(Δq1−Δq2)/Δt [5]

where:

Kp is a proportional spring constant;

Kd is a derivative spring constant;

q1 is the joint angle of the first input handle 42a;

q1 is the joint angle of the second input handle 42b; and
t is a time.

By including the second portion of equation 5, the difference in velocity of the joint angles of each input handle 42a, 42b are taken between two time instances, which may reduce the possibility of oscillation of the motion of the second input handle 42b during operation.

In considering the use of each of these equations, it should be appreciated by those skilled in the art that equations 4 and 5 are simplifications.

In still another embodiment, a location of where to position the input handles 42a, 42b is based on the position of the tool 20. For example, in an embodiment, a position of the tool 20 is obtained relative to a base 61 of the robotic arm 18 to which the tool 20 is coupled. In another embodiment, the position of the tool 20 is obtained from an image of the surgical site “S” acquired by the imaging device 56.

When an input is received representing a movement of the first input handle 42a, a signal representing the input is transmitted to the controller 230. In response to receiving the signal, the controller 230 sends commands to the tool 20 to effect movement thereof to an intended location. The intended location is represented as a vector or an x-y-z coordinate. In an embodiment, the location of the tool 20 is also or alternatively determined, for example, as a coordinate. The controller 230 translates the intended location or the coordinate of the tool 20 into a suitable position for the second input handle 42b and sends a signal to the second console 240b to output a force to the second input handle 42b to thereby move the second input handle 42b to the position. The force output is determined by determining a difference between the translated position of the tool 20 and the intended location of the tool 20 (effected by the first input handle 42a), then multiplying the difference by a spring constant and adding a force exerted by the system 1.

The above description is represented by the following equations:

Fₓ₁=F_{sys}+k₁(\vec{X}₁−\vec{X}₂) [6]

Fₓ₂=F_{sys}+k₂(\vec{X}2-\vec{X}1) [7]

where:

F_{sys} is the amount of force provided by the system, including forces, such as force feedback, collision forces, range of motion limits or virtual constraints, and/or boundary limits of the system;

k₁ is a spring constant of the first input handle;

k₂ is a spring constant of the second input handle;

\vec{X}₁ is the position of the surgical tool as received from the first input handle (desired position);

\vec{X}₂ is the position of the surgical tool as received from the second input handle;

Fₓ₁ is the amount of force needed to move the first input handle to the desired position; and

Fₓ₂ is the amount of force needed to move the second input handle to the desired position.

In an embodiment, the system 1 operates in or a non-lock out mode or does not include a lock-out mode and is further configured to act in response to the input provided by the novice clinician at the second console 240b. For example, the controller 320 detects a movement of the second input handle 42b in a direction that is not substantially identical to a movement of the first input handle 42a, and in response to detecting the movement of the second input handle 42b, the controller 320 provides a signal to increase a stiffness output by the second input handle 42b. In accordance with an embodiment, the stiffness output by the second input handle 42b increases based on an increase in a distance between an intended path of the first input handle 42a and a movement path of the second input handle 42a. As a result, moving the second input handle 42b becomes more difficult the further away its path deviates from the intended path of the first input handle 42a. In another embodiment, the controller 320 detects a movement of the second input handle 42b in a direction that is not substantially identical to a movement of the first input handle 42a, and in response to detecting the movement of the second input handle, a torque output by the second input handle 42b increases.

According to another embodiment in which the system 1 operates in a non-lock out mode or does not include the lock-out mode, input provided to the second console 240b affects movement of the selected portion of the robotic arm. For example, in an embodiment, the system 1 is configured to be selectively placed into a dual input mode in which the expert clinician and the novice clinician can both provide inputs to the consoles 240a, 240b and effect movement of the robotic arms, instruments, and/or tools. In this regard, the novice clinician can drive the robotic arm, if the expert clinician does not provide input.

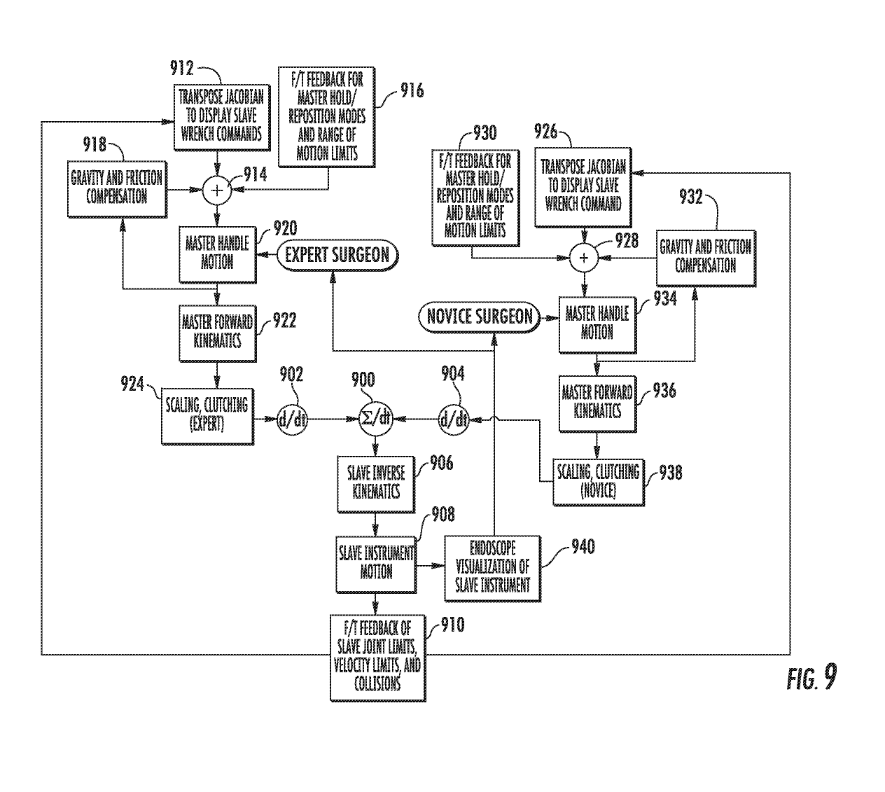

FIG. 9 is a flow diagram illustrating operation of the system 1 in a non-lock out mode, according to an embodiment. Beginning at block 900, an input to effect a desired instrument pose provided by the expert clinician over a time period at block 902 is added to an input of a desired instrument pose provided by the novice clinician over the same time period at block 904. In each of blocks 902 and 904, derivatives are calculated of the pose of the input handles 42a, 42b to yield various aspects of the poses, such as a velocity of each, roll, pitch, yaw, and instrument jaw angle for each input handle 42a, 42b, and the like. The velocities and roll, pitch, yaw, and instrument jaw angles of each input handle 42a, 42b are added, and the result of block 900 outputs the desired instrument pose.

Slave inverse kinematics are then calculated at block 906 outputting the desired slave joint angles (for example, the joint angles at which the tool 20 to be controlled is positioned). The desired slave joint angles are used to then effect the slave instrument motion at block 908. The slave instrument motion is used for endoscope visualization of the slave instrument at block 940. As such, images of the tool 20 are processed into a three-dimensional high definition video feed, which is provided to the consoles 240a, 240b for display. The slave instrument motion is further used in outputting actual joint angles. The F/T feedback is then calculated at block 910 based on the output actual slave joint angles. As noted above, force feedback can be used to provide a haptic indication of the state of the slave robot and instrument under control, such as when the slave robot or instrument reaches joint range of motion limits, velocity limits, and collisions. Similar to above, the F/T feedback of the slave joint limits, velocity limits, and collisions may be predetermined values that may be pre-set or set by the expert clinician as desired parameters.

The F/T wrench output is then processed at each of the consoles 240a, 240b. For example, on the expert clinician side, a transpose Jacobian is used to calculate required joint torques in the input device to display slave wrench commands from block 910, which are combined at block 914 with joint torques required for hold/reposition modes and range of motion limits (which may be predetermined values that may be pre-set or set by the expert clinician as desired parameters) from block 916, along with a gravity and friction compensation from block 918 (obtained when the master handle motion was performed at block 920). As a result of the combined operation, joint torques are output at block 914, which are taken into account at block 920, when a master handle motion is effected by the expert clinician. Joint angles of the master input device are measured from the input to allow forward kinematics to be obtained at block 922, which outputs the desired master handle pose. The master handle pose from block 922 is provided to block 942, where scaling and clutching is applied at block 942. The scaling and clutching may be pre-set by the expert clinician, depending on the expert clinician's preference or may be included as a factory-installed parameter. A derivative of the output from block 924 is calculated at block 902 providing an instrument pose intended by the input provided to the first input handle 42a, which as noted above, is then used in the calculation at block 900.

The operations in the non-lock out mode from the viewpoint of the novice clinician is similar to those described above with respect to the expert clinician. On the novice clinician side, a transpose Jacobian is applied to the F/T wrench output from block 910 to calculate the required joint torques in the input device to display the desired slave wrench commands at block 926, and the output of block 926 is combined with the joint torques required for hold/reposition modes and range of motion limits (may be predetermined values that may be pre-set or set by the expert clinician as desired parameters) from block 930, and gravity and friction compensation from block 920 (calculated using the joint angles output from block 934), which are obtained when a master handle motion is effected by the expert clinician at block 934 to yield the joint torques for the second input handle 42b at block 928. Joint angles are measured from block 934 from the input to allow forward kinematics to be obtained at block 936, which outputs the master handle pose. Scaling and clutching is applied to the master handle pose at block 938. Here, the scaling and clutching may be pre-set by the expert clinician, depending on the novice clinician's experience or ability. For example, a novice clinician having very little experience may need larger scaling and/or clutching where large inputs to the second input handle 42b translate into small movements by the tool 20, while a novice clinician having more experience may need less scaling and/or clutching. In another embodiment, the scaling and clutching may be set as a factory-installed parameter. A derivative of the output from block 938 is taken at block 904 providing an instrument pose intended by the input provided to the second input handle 42b, which is then used in the calculation at block 900.

In the aforementioned embodiment, the system 1 further may include an override mode. For example, in response to the controller 320 detecting a movement of the second input handle 42b by the novice clinician in a direction that is not substantially identical to a movement of the first input handle 42a being manipulated by the expert clinician, signals are sent from the controller 320 to override the movement of the second input handle 42b to thereby move the robotic arm according to the movement of the first input handle 42a. In an embodiment, overriding commands are sent from the controller 320 to the second console 240b, for example, canceling and adding to any input commands from the second console 240b. In another embodiment, overriding commands are sent from the controller 320 to the robotic arm, canceling or blocking any input commands from the second console 240b.

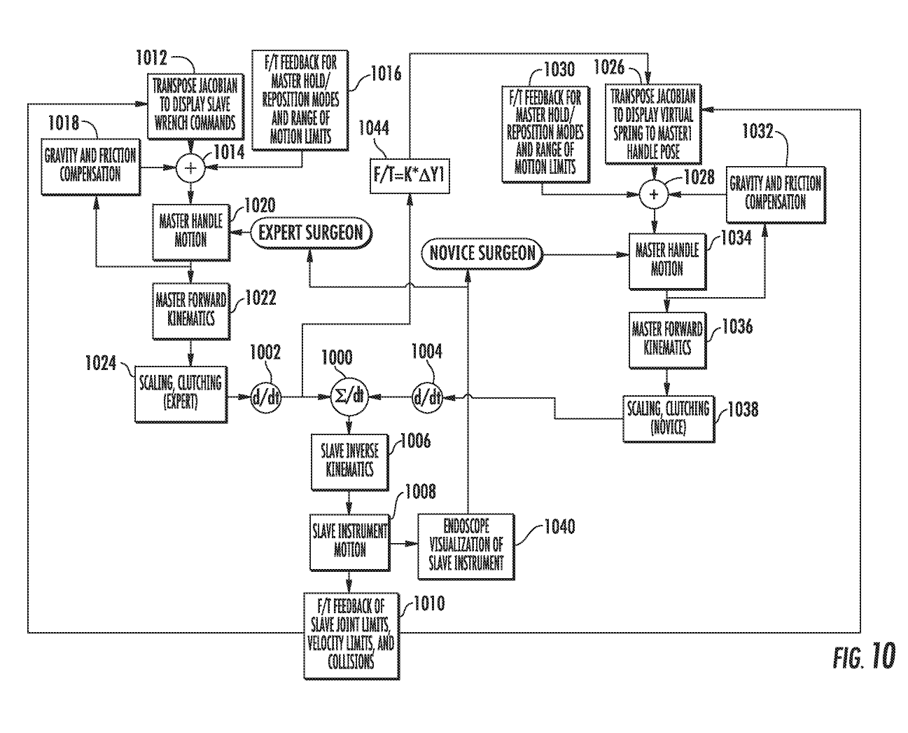

FIG. 10 is a flow diagram illustrating operation of the system 1 in a non-lock out mode, according to another embodiment. Here, the force/torque provided by the expert clinician influences the handle motion at the novice clinician's console. The operations of blocks 1000 through 1040 are identical to those of blocks 900 through 940 of FIG. 9, except the desired instrument pose of the first input handle 42a (ΔY1), which is output from block 1002, is used at block 1044 to provide a force/torque to the second input handle 42b. A representative equation for taking into account the additional factor is provided as the following equation:

F/T=K*ΔY1 [8]

where:

K is a spring constant; and

ΔY1 is the desired instrument pose of the first input handle 42a.

It will be appreciated that equation 8 is a simplification and other equations may be used to calculate other aspects that may need to be taken into account. A transpose Jacobian function is then applied to the output of block 1044 at block 1026 and the method continues.

As briefly discussed above with regard to scaling and clutching, to provide further control for the expert clinician while training the novice clinician, in accordance with the embodiment, the responsiveness of the movement of the robotic arms, instruments and/or tools are different depending on which console 240a, 240b from which an input originates. For example, the signals provided from each console 240a, 240b may be scaled differently. In an embodiment, when the expert clinician is located at a first console 240a, the expert clinician moves the input handles 42a a first distance in order to effect a movement of the instrument 22 or tool 20. When the novice clinician is located at the second console 240b, the novice clinician moves the input handles 42b a second distance that is greater than the first distance in order to effect the same movement of the instrument 22 or tool 20. The scaling of the input into the two consoles 240a, 240b is taken into account primarily in embodiments in which the user interfaces 40 and consoles 240a, 240b are substantially identical in configuration.

In any case, in order to provide scaling in the system 10, the processing unit 30 transmits scaled control signals to the robot base 18 to move the robotic arms 236a-d and tools 20 in response to the movement of the input handles 42a, 24b such that the movement of the robotic arms 236a-d and tools 20 are scaled depending on from which console 240a, 240b an input is received. For example, the expert clinician at the first console 240a may provide input to the first input handle 42a, while the novice clinician at the second console 240b provides input to input handle 42b, and the control signal from each of the consoles 240a, 240b to the robot base 18 is scaled to a particular factor. The equation below represents how inputs between the two consoles 240a, 24b are scaled:

ΔXₛ=s₁Δx₁+s₂Δx₂ [9]

where:
s₁ is a selected scaling factor assigned to console 1;

s₂ is a selected scaling factor assigned to console 2;

Δx₁ is a distance the input handle at console 1 moves;

Δx₂ is a distance the input handle at console 2 moves; and

ΔXₛ is a change in desired position.

In embodiments in which the user interfaces 40 and consoles 240a, 240b are not substantially identically configured, scaling is independently considered.

The systems described herein may also utilize one or more controllers to receive various information and transform the received information to generate an output. The controller may include any type of computing device, computational circuit, or any type of processor or processing circuit capable of executing a series of instructions that are stored in a memory. The controller may include multiple processors and/or multicore central processing units (CPUs) and may include any type of processor, such as a microprocessor, digital signal processor, microcontroller, or the like. The controller may also include a memory to store data and/or algorithms to perform a series of instructions.

Any of the herein described methods, programs, algorithms or codes may be converted to, or expressed in, a programming language or computer program. A “Programming Language” and “Computer Program” includes any language used to specify instructions to a computer, and includes (but is not limited to) these languages and their derivatives: Assembler, Basic, Batch files, BCPL, C, C+, C++, Delphi, Fortran, Java, JavaScript, Machine code, operating system command languages, Pascal, Perl, PL1, scripting languages, Visual Basic, metalanguages which themselves specify programs, and all first, second, third, fourth, and fifth generation computer languages. Also included are database and other data schemas, and any other meta-languages. No distinction is made between languages which are interpreted, compiled, or use both compiled and interpreted approaches. No distinction is also made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

Any of the herein described methods, programs, algorithms or codes may be contained on one or more machine-readable media or memory. The term “memory” may include a mechanism that provides (e.g., stores and/or transmits) information in a form readable by a machine such a processor, computer, or a digital processing device. For example, a memory may include a read only memory (ROM), random access memory (RAM), magnetic disk storage media, optical storage media, flash memory devices, or any other volatile or non-volatile memory storage device. Code or instructions contained thereon can be represented by carrier wave signals, infrared signals, digital signals, and by other like signals.

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as exemplifications of particular embodiments. Those skilled in the art will envision other modifications within the scope of the claims appended hereto.

1. A robotic surgical system, comprising:
a first console including a first input handle;
a second console including a second input handle;
a robotic arm; and
a controller coupled to the first console, the second console, and the robotic arm, the controller including:
a processor, and
a memory coupled to the processor, the memory storing instructions that, when executed by the processor, cause the controller to:
receive an input from the first console via the first input handle to cause the robotic arm to move;
in response to receiving the input from the first input handle, move the robotic arm; and
substantially simultaneously with the moving of the first input handle, provide an output to thereby cause the second input handle of the second console to move in substantially the same motion as the first input handle.

2. The robotic surgical system of claim 1, further comprising instructions that, when executed by the processor, cause the controller to provide a signal to the second console to provide a force feedback, in response to receiving an input from the second input handle.

3. The robotic surgical system of claim 1, wherein the first console and the second console are substantially identically configured.

4. The robotic surgical system of claim 3, wherein each of the first console and the second console has a corresponding base, and positioning of each of the input handles of each of the first console and the second console is based on a fixed coordinate frame relative to its corresponding base.

5. The robotic surgical system of claim 3, wherein each of the first console and the second console has a plurality of support arms, and positioning of the input handles of the each of the first console and the second console is based on positioning of a first support arm of the first console relative to a second support arm of the first console and a first support arm of the second console relative to a second support arm of the second console.

6. The robotic surgical system of claim 1, wherein the first console and the second console are not substantially identically configured and wherein each of the first console and the second console has an input handle, and positioning of each of the input handles of each of the first console and the second console is based on Cartesian coordinates of each of the input handles.

7. The robotic surgical system of claim 1, further comprising instructions that, when executed by the processor, cause the controller to:
detect a movement of the second input handle in a direction that is not substantially identical to a movement of the first input handle; and
in response to detecting the movement of the second input handle, increase a stiffness output by the second input handle.

8. The robotic surgical system of claim 7, wherein the stiffness output increases based on an increase in a distance between a movement path of the first input handle and a movement path of the second input handle.

9. The robotic surgical system of claim 1, further comprising instructions that, when executed by the processor, cause the controller to:
detect a movement of the second input handle in a direction that is not substantially identical to a movement of the first input handle; and
in response to detecting the movement of the second input handle, increase a torque output by the second input handle.

10. The robotic surgical system of claim 1, wherein:
the memory further comprises instructions that, when executed by the processor, cause the controller to:
detect a movement of the second input handle in a direction that is not substantially identical to a movement of the first input handle; and
in response to detecting the movement of the second input handle, overriding the movement of the second input handle to thereby move the robotic arm according to the movement of the first input handle.

11. A method of operating a dual console robotic surgical system, the method comprising:
receiving an input from a first console of the dual console robotic surgical system via a first input handle to move a robotic arm of the robotic surgical system;
in response to receiving the input from the first input handle, moving the robotic arm; and
substantially simultaneously with the moving of the first input handle, providing an output to thereby move a second input handle of a second console of the dual console robotic surgical system in substantially the same motion as the first input handle.

12. The method of claim 11, further comprising providing a signal to the second console of the dual console robotic surgical system to provide a force feedback, in response to receiving an input from the second input handle.

13. The method of claim 11, further comprising:
detecting a movement of the second input handle in a direction that is not substantially identical to a movement of the first input handle; and
in response to detecting the movement of the second input handle, increasing a stiffness output by the second input handle.

14. The method of claim 13, further comprising:
increasing the stiffness output increases based on an increase in a distance between a movement path of the first input handle and a movement path of the second input handle.

15. The method of claim 11, further comprising:
detecting a movement of the second input handle in a direction that is not substantially identical to a movement of the first input handle; and
in response to detecting the movement of the second input handle, increasing a torque output by the second input handle.

16. The method of claim 11, wherein the first console is a main console and the second console is an auxiliary console.

17. The method of claim 11, further comprising
detecting a movement of the second input handle in a direction that is not substantially identical to a movement of the first input handle; and
in response to detecting the movement of the second input handle, overriding the movement of the second input handle to thereby move the robotic arm according to the movement of the first input handle.

18-19. (canceled)

20. A robotic surgical system, comprising:
a first console including a first input handle;
a second console including a second input handle;
a robotic arm including a surgical tool configured to be disposed adjacent to a surgical site; and
a controller coupled to the first console, the second console, and the robotic arm, the controller including:
a processor, and
a memory coupled to the processor, the memory storing instructions that, when executed by the processor, cause the controller to:
receive an input from the first console via the first input handle to move the surgical tool to a location within the surgical site;
determine a coordinate of the location of the surgical tool within the surgical site; and
provide an output to move the second input handle of the second console to a position that translates to the location of the surgical tool within the surgical site.

21. The robotic surgical system of claim 20, wherein the memory further includes instructions that, when executed by the processor, cause the controller to obtain a position of the surgical instrument relative to a base of the robotic arm to which the surgical instrument is coupled.

22. The robotic surgical system of claim 20, further comprising an imaging device coupled to the controller, the imaging device configured to be disposed over the surgical site, wherein the memory further includes instructions that, when executed by the processor, cause the controller to obtain a position of the surgical instrument from an image of the surgical site acquired by the imaging device.
